(19) United States
(12) Patent Application Publication
(10) Pub. No.: US 2012/0149903 A1
(43) Pub. Date: Jun. 14, 2012

(54) PROCESS FOR PREPARING AND PURIFYING 3-AMINOPROPANOL

(75) Inventors: Manfred Knoll, Ludwigshafen (DE); Andreas Edgar Herrmann, Speyer (DE); Dominik Herbrecht, Heidelberg (DE)

(73) Assignee: BASF SE, Ludwigshafen (DE)

(21) Appl. No.: 13/325,594

(22) Filed: Dec. 14, 2011

Related U.S. Application Data
(60) Provisional application No. 61/422,673, filed on Dec. 14, 2010.

(30) Foreign Application Priority Data
Dec. 14, 2010 (EP) 10194921.2

(51) Int. Cl. C07D 403/12; C07C 231/10; B01D 3/36; C07C 209/68; C07D 401/14; C07F 9/6584; C07C 213/02; C07C 303/02

(52) U.S. Cl. 544/295; 564/493; 564/203; 562/105; 564/381; 546/199; 558/81; 203/32

(57) ABSTRACT

The present invention relates to a process for purifying a reaction output which comprises 3-aminopropanol and is obtained in the reaction of ethylene cyanohydrin with hydrogen in the presence of ammonia, which comprises distilling the reaction output comprising 3-aminopropanol in two or more stages, the ammonia content of the reaction output comprising 3-aminopropanol before introduction into the first distillation stage being 1% by weight or less and the temperature in the distillation stages being not more than 135° C.

The invention further relates to a process for preparing 3-aminopropanol by reacting ethylene cyanohydrin with hydrogen in the presence of ammonia, which comprises performing the purification of the reaction output comprising 3-aminopropanol in accordance with the invention. The present invention further provides a process for preparing 3-aminopropanol derivatives, especially panthenol, acambrosate, mefenorex, domperidon, ifosamid or urapidil, from 3-amino-propanol prepared in accordance with the invention.

The present application incorporates the provisional U.S. Application 61/422,673 filed Dec. 14, 2010 by reference.

The present invention relates to a process for purifying a reaction output which comprises 3-aminopropanol and is obtained in the reaction of ethylene cyanohydrin with hydrogen in the presence of ammonia. The invention further relates to the preparation of 3-aminopropanol and to the use thereof. The present invention further provides a process for preparing 3-aminopropanol derivatives, especially panthenol, which comprises using a 3-aminopropanol which has been purified in accordance with the invention in the preparation of the 3-aminopropanol derivatives.

3-Aminopropanol is typically prepared by reacting ethylene cyanohydrin with hydrogen.

German patent 573983 discloses the hydrogenation of ethylene cyanohydrin in the presence of hydrogenation catalysts of groups 8, 9 and 10 of the periodic table of the elements. After the hydrogenation, the reaction product is removed from the catalyst and purified by fractional distillation.

CH-B-244837 describes the catalytic reduction of nitriles, including ethylene cyanohydrin, which have been dissolved or suspended in liquid ammonia and then catalytically hydrogenated under pressure. According to the disclosure, the use of liquid ammonia suppresses the formation of secondary bases, such that primary amine forms as the main product in the hydrogenation. After the hydrogenation has ended, ammonia is distilled off, and the reaction product is separated from the catalyst and then distilled under reduced pressure.

DE-B-2655794 discloses a further process for preparing 3-aminopropanol. In a preferred embodiment of the process, after the ethylene cyanohydrin synthesis, the product obtained is reductively aminated. Ammonia is used in an excess of 10 to 30 mol per mole of ethylene cyanohydrin. The reduction is performed with hydrogen in the presence of a hydrogenation catalyst. After the end of the reaction, the reaction mixture is cooled and optionally filtered. Aminopropanol is removed from the filtrate by distillation under reduced pressure.

European patent application EP-A1-1132371 describes a process for preparing alkanolamines, including 3-aminopropanol, with improved color quality, in which the alkanolamines are distilled or rectified in the presence of a phosphorus compound under reduced pressure.

A further process for catalytic hydrogenation of ethylene cyanohydrin is detailed in JP-A-2002201164. The hydrogenation is performed in the presence of a Raney cobalt catalyst and ammonia, which suppressed the formation of secondary and tertiary amines, such that it was possible to obtain pure aminopropanol by simple distillation.

JP-A-2002053535 describes the distillation of aminopropanol in the presence of tetrahydroborates in order to obtain high-purity aminopropanol with low proportions of morpholines and morpholine derivatives.

Japanese patent application JP-A-05163213, in contrast, discloses the use of Raney cobalt catalysts in order to achieve 3-aminopropanol with improved yield.

3-Aminopropanol is an important starting material for the production of cosmetics, pharmaceuticals and crop protection compositions. The demands on quality and purity are therefore very high. More particularly, for 3-aminopropanol which for the preparation of panthenol and panthenol derivates which are used as a constituent of ointments in cosmetics and for medical applications, there is a high requirement on the odor. The 3-aminopropanol used may have only a slight intrinsic odor since the ointments are generally applied directly to the human skin, and an intrinsic odor would not be accepted by many consumers.

It has now been found that 3-aminopropanol which has been purified by conventional processes, such as distillation or rectification, does not meet the strict quality demands of many consumers in the cosmetic and pharmaceutical industry, since it has too strong an intrinsic odor.

The object of the present invention consisted in the provision of a process for purifying 3-aminopropanol to obtain a high-purity 3-aminopropanol which has low intrinsic odor compared to the prior art and meets the quality standards of the cosmetic and pharmaceutical industry.

The object was achieved in accordance with the invention by a process for purifying a reaction output which comprises 3-aminopropanol and is obtained in the reaction of ethylene cyanohydrin with hydrogen in the presence of ammonia, which comprises distilling the reaction output comprising 3-aminopropanol in two or more stages, the ammonia content of the reaction output comprising 3-aminopropanol before introduction into the first distillation stage being 1% by weight or less and the temperature in the distillation stages being not more than 135° C.

The present invention further provides a process for preparing 3-aminopropanol by reacting ethylene cyanohydrin with hydrogen in the presence of ammonia, which comprises performing the purification of the 3-aminopropanol in accordance with the invention.

3-Aminopropanol is obtained by reacting ethylene cyanohydrin with hydrogen in the presence of ammonia.

Preference is given to reacting 3-aminopropanol with hydrogen and ammonia in the presence of a catalyst.

Ethylene cyanohydrin is used in the process according to the invention.

Ethylene cyanohydrin can be prepared via various preparation routes.

In the process according to the invention, preference is given to using ethylene cyanohydrin which has been prepared by reaction of ethylene oxide with hydrogen cyanide.

Such ethylene cyanohydrin is obtained, for example, as an intermediate in the preparation of acrylonitrile. In acrylonitrile preparation, ethylene oxide is generally reacted with hydrogen cyanide in a basic environment to give ethylene cyanohydrin, which can be converted further to acrylonitrile with the elimination of a water molecule over an Al₂O₃ catalyst.

Before the reaction with hydrogen in the presence of ammonia, ethylene cyanohydrin can be purified, for example by distillation, but it can also be used directly in the hydrogenation—without further workup.

Hydrogen is used in the process according to the invention.

The hydrogen is generally used in industrial purity. The hydrogen can also be used in the form of a hydrogen-comprising gas, i.e. in additions with other inert gases, such as nitrogen, helium, neon, argon or carbon dioxide. The hydrogen-comprising gases used may, for example, be reformer offgases, refinery gases, etc., if and to the extent that these gases do not comprise any catalyst poisons for the hydrogenation catalysts used, for example CO. Preference is given, however, to using pure hydrogen or essentially pure hydrogen in the process, for example hydrogen with a content of more than 99% by weight of hydrogen, preferably more than 99.9% by weight of hydrogen, more preferably more than 99.99% by weight of hydrogen, especially more than 99.999% by weight of hydrogen.

Ammonia is also used in the process according to the invention.

The ammonia used may be conventional, commercially available ammonia, for example ammonia with a content of more than 98% by weight of ammonia, preferably more than 99% by weight of ammonia, preferably more than 99.5% by weight, especially more than 99.9% by weight of ammonia.

Ethylene cyanohydrin is reacted with ammonia and hydrogen in the presence of a catalyst.

The catalysts used to hydrogenate the nitrile function of the cyanohydrin to give aminopropanol may especially be catalysts which comprise, as the active component, one or more elements of transition group 8 of the periodic table (Fe, Co, Ni, Ru, Rh, Pd, Os, Ir, Pt), preferably Fe, Co, Ni, Ru or Rh, more preferably Co or Ni.

The abovementioned catalysts can be doped in a customary manner with promoters, for example with chromium, iron, cobalt, manganese, molybdenum, titanium, tin, metals of the alkali metal group, metals of the alkaline earth metal group and/or phosphorus.

The catalysts used may be what are called skeletol catalysts (also known as the Raney® type, hereinafter also: Raney catalyst), which are obtained by leaching (activating) an alloy composed of hydrogenation-active metal and a further component (preferably Al). Preference is given to using Raney nickel catalysts or Raney cobalt catalysts.

However, the catalysts used may also be catalysts which have been obtained by reduction of what are called oxidic catalyst precursors.

In a preferred embodiment, catalysts which are prepared by reduction of what are called catalyst precursors are used in the process according to the invention.

The catalyst precursor comprises an active material which comprises one or more catalytically active components, optionally promoters and optionally a support material.

The catalytically active components are oxygen compounds of the abovementioned metals, for example the metal oxides or hydroxides thereof, such as CoO, NiO, CuO and/or mixed oxides thereof.

In the context of this application, the term “catalytically active components” is used for abovementioned oxygen-metal compounds, but is not intended to imply that these oxygen compounds are already catalytically active per se. The catalytically active components generally have catalytic activity in the inventive conversion only on completion of reduction.

The catalyst precursors can be prepared by known processes, for example by precipitation, precipitative application or impregnation.

In a preferred embodiment, catalyst precursors which are prepared by impregnating support materials are used in the process according to the invention (impregnated catalyst precursors). The support materials used in the impregnation can, for example, be used in the form of powders or shaped bodies, such as extrudates, tablets, spheres or rings. Support material suitable for fluidized bed reactors is preferably obtained by spray drying.

Useful support materials include, for example, carbon such as graphite, carbon black and/or activated carbon, aluminum oxide (gamma, delta, theta, alpha, kappa, chi or mixtures thereof), silicon dioxide, zirconium dioxide, zeolites, aluminosilicates or mixtures thereof.

The abovementioned support materials can be impregnated by the customary methods (A. B. Stiles, Catalyst Manufacture—Laboratory and Commercial Preparations, Marcel Dekker, New York, 1983), for example by applying a metal salt solution in one or more impregnation stages.

Useful metal salts generally include water-soluble metal salts, such as the nitrates, acetates or chlorides of the corresponding catalytically active components or the doping elements, such as cobalt nitrate or cobalt chloride. Thereafter, the impregnated support material is generally dried and optionally calcined.

The calcination is generally performed at temperatures between 300 and 800° C., preferably 350 to 600° C., especially at 450 to 550° C.

The impregnation can also be effected by the so-called “incipient wetness method”, in which the support material is moistened with the impregnating solution up to a maximum of saturation according to its water absorption capacity. However, the impregnation can also be effected in supernatant solution.

In the case of multistage impregnation processes, it is appropriate to dry and if appropriate to calcine between individual impregnation steps. Multistage impregnation can be employed advantageously when the support material is to be contacted with metal salts in a relatively large amount.

To apply a plurality of metal components to the support material, the impregnation can be effected simultaneously with all metal salts or in any desired sequence of the individual metal salts.

In a further preferred embodiment, catalyst precursors are prepared by means of a coprecipitation of all of their components. To this end, in general, a soluble compound of the corresponding active component and of the doping elements, and optionally a soluble compound of a support material are admixed with a precipitant in a liquid while heating and while stirring until the precipitation is complete.

The liquid used is generally water.

Useful soluble compounds of the active components typically include the corresponding metal salts, such as the nitrates, sulfates, acetates or chlorides of the aforementioned metals.

The soluble compounds of a support material used are generally water-soluble compounds of Ti, Al, Zr, Si etc., for example the water-soluble nitrates, sulfates, acetates or chlorides of these elements.

The soluble compounds of the doping elements used are generally water-soluble compounds of the doping elements, for example the water-soluble nitrates, sulfates, acetates or chlorides of these elements.

Catalyst precursors can also be prepared by precipitative application.

Precipitative application is understood to mean a preparation method in which a sparingly soluble or insoluble support material is suspended in a liquid and then soluble compounds, such as soluble metal salts, of the appropriate metal oxides, are added, which are then precipitated onto the suspended support by adding a precipitant (for example, described in EP-A2-1 106 600, page 4, and A. B. Stiles, Catalyst Manufacture, Marcel Dekker, Inc., 1983, page 15).

Useful sparingly soluble or insoluble support materials include, for example, carbon compounds such as graphite, carbon black and/or activated carbon, aluminum oxide (gamma, delta, theta, alpha, kappa, chi or mixtures thereof), silicon dioxide, zirconium dioxide, zeolites, aluminosilicates or mixtures thereof.

The support material is generally present in the form of powder or spall.

The liquid used, in which the support material is suspended, is typically water.

Useful soluble compounds include the aforementioned soluble compounds of the active components or of the doping elements.

Typically, in the precipitation reactions, the soluble compounds are precipitated as sparingly soluble or insoluble basic salts by adding a precipitant.

The precipitants used are preferably alkalis, especially mineral bases, such as alkali metal bases. Examples of precipitants are sodium carbonate, sodium hydroxide, potassium carbonate or potassium hydroxide.

The precipitants used may also be ammonium salts, for example ammonium halides, ammonium carbonate, ammonium hydroxide or ammonium carboxylates.

The precipitation reactions can be performed, for example, at temperatures of 20 to 100° C., preferably 30 to 90° C., especially at 50 to 70° C.

The precipitates formed in the precipitation reactions are generally chemically inhomogeneous and generally comprise mixtures of the oxides, oxide hydrates, hydroxides, carbonates and/or hydrogencarbonates of the metals used. It may be found to be favorable for the filterability of the precipitates when they are aged, i.e. when they are left alone for a certain time after the precipitation, if appropriate under hot conditions or while passing air through.

The precipitates obtained by these precipitation processes are typically processed by washing, drying, calcining and conditioning them.

After washing, the precipitates are generally dried at 80 to 200° C., preferably 100 to 150° C., and then calcined.

The calcination is performed generally at temperatures between 300 and 800° C., preferably 350 to 600° C., especially at 450 to 550° C.

After the calcination, the pulverulent catalyst precursors obtained by precipitation reactions are typically conditioned.

The conditioning can be effected, for example, by adjusting the precipitation catalyst to a particular particle size by grinding.

After the grinding, the catalyst precursor obtained by precipitation reactions can be mixed with shaping assistants such as graphite or stearic acid, and processed further to shaped bodies.

Common processes for shaping are described, for example, in Ullmann [Ullmann's Encyclopedia Electronic Release 2000, chapter: “Catalysis and Catalysts”, pages 28-32] and by Ertl et al. [Ertl, Knozinger, Weitkamp, Handbook of Heterogeneous Catalysis, VCH Weinheim, 1997, pages 98 ff].

As described in the references cited, the process for shaping can provide shaped bodies in any three-dimensional shape, for example round, angular, elongated or the like, for example in the form of extrudates, tablets, granules, spheres, cylinders or grains. Common processes for shaping are, for example, extrusion, tableting, i.e. mechanical pressing, or pelletizing, i.e. compacting by circular and/or rotating motions.

The conditioning or shaping is generally followed by a heat treatment. The temperatures in the heat treatment typically correspond to the temperatures in the calcination.

The catalyst precursors obtained by precipitation reactions or impregnation comprise the catalytically active components in the form of a mixture of oxygen compounds thereof, i.e. especially as the oxides, mixed oxides and/or hydroxides. The catalyst precursors thus prepared can be stored as such.

Particular preference is given to catalyst precursors such as

the oxide mixtures which are disclosed in EP-A-0636409 and which comprise, before the reduction with hydrogen, 55 to 98% by weight of Co, calculated as CoO, 0.2 to 15% by weight of phosphorus, calculated as H₃PO₄, 0.2 to 15% by weight of manganese, calculated as MnO₂, and 0.2 to 5.0% by weight of alkali metal, calculated as M₂O (M=alkali metal), or

oxide mixtures which are disclosed in EP-A-0742045 and which comprise, before the reduction with hydrogen, 55 to 98% by weight of Co, calculated as CoO, 0.2 to 15% by weight of phosphorus, calculated as H₃PO₄, 0.2 to 15% by weight of manganese, calculated as MnO₂, and 0.05 to 5% by weight of alkali metal, calculated as M₂O (M=alkali metal), or oxide mixtures which are disclosed in EP-A-696572 and which comprise, before the reduction with hydrogen, 20 to 85% by weight of ZrO₂, 1 to 30% by weight of oxygen compounds of copper, calculated as CuO, 30 to 70% by weight of oxygen compounds of nickel, calculated as NiO, 0.1 to 5% by weight of oxygen compounds of molybdenum, calculated as MoO₃, and 0 to 10% by weight of oxygen compounds of aluminum and/or manganese, calculated as Al₂O₃ and MnO₂ respectively, for example the catalyst disclosed in loc. cit., page 8, with the composition of 31.5% by weight of ZrO₂, 50% by weight of NiO, 17% by weight of CuO and 1.5% by weight of MoO₃, or

oxide mixtures which are disclosed in EP-A-963 975 and which comprise, before the reduction with hydrogen, 22 to 40% by weight of ZrO2, 1 to 30% by weight of oxygen compounds of copper, calculated as CuO, 15 to 50% by weight of oxygen compounds of nickel, calculated as NiO, where the molar Ni:Cu ratio is greater than 1, 15 to 50% by weight of oxygen compounds of cobalt, calculated as CoO, 0 to 10% by weight of oxygen compounds of aluminum and/or manganese, calculated as Al₂O₃ and MnO₂ respectively, and no oxygen compounds of molybdenum, for example the catalyst A disclosed in loc. cit., page 17, with the composition of 33% by weight of Zr, calculated as ZrO₂, 28% by weight of Ni, calculated as NiO, 11% by weight of Cu, calculated as CuO and 28% by weight of Co, calculated as CoO, or

The catalyst precursors which have been prepared as described above by impregnation or precipitation are generally reduced after the calcination or conditioning. The reduction generally converts the catalyst precursor to its catalytically active form.

The reduction of the catalyst precursor can be performed at elevated temperature in a moving or stationary reduction oven.

The reducing agent used is typically hydrogen or a hydrogen-comprising gas.

The hydrogen is generally used in technical grade purity. The hydrogen can also be used in the form of a hydrogen-comprising gas, i.e. in admixtures with other inert gases, such as nitrogen, helium, neon, argon or carbon dioxide. The hydrogen stream can also be recycled into the reduction as cycle gas, optionally mixed with fresh hydrogen and optionally after removing water by condensation.

The catalyst precursor is preferably reduced in a reactor in which the shaped catalyst bodies are arranged as a fixed bed. The catalyst precursor is more preferably reduced in the same reactor in which the subsequent reaction of ethylene cyanohydrin with ammonia is effected.

In addition, the catalyst precursor can be reduced in a fluidized bed reactor in the fluidized bed.

The catalyst precursor is generally reduced at reduction temperatures of 50 to 600° C., especially of 100 to 500° C., more preferably of 150 to 450° C.

The partial hydrogen pressure is generally from 1 to 300 bar, especially from 1 to 200 bar, more preferably from 1 to 100 bar, where the pressure figures here and hereinafter are based on the absolute measured pressure.

The duration of the reduction is preferably 1 to 20 hours and more preferably 5 to 15 hours.

During the reduction, a solvent can be supplied in order to remove water of reaction which forms and/or in order, for example, to be able to heat the reactor more rapidly and/or to be able to better remove the heat during the reduction. In this case, the solvent can also be supplied in supercritical form.

Suitable solvents used may be the above-described solvents. Preferred solvents are water; ethers such as methyl tert-butyl ether, ethyl tert-butyl ether, dioxane or tetrahydrofuran. Particular preference is given to water or tetrahydrofuran. Suitable solvents likewise include suitable mixtures.

The catalyst precursor can also be reduced in suspension, for example in a stirred autoclave. The temperatures are generally within a range from 50 to 300° C., especially from 100 to 250° C., more preferably from 120 to 200° C.

The reduction in suspension is generally performed at a partial hydrogen pressure of 1 to 300 bar, preferably from 10 to 250 bar, more preferably from 30 to 200 bar. Useful solvents include the aforementioned solvents.

The duration of the reduction in suspension is preferably 5 to 20 hours, more preferably 8 to 15 hours.

The catalyst thus obtained can be handled under inert conditions after the reduction. The catalyst can preferably be handled and stored under an inert gas such as nitrogen, or under an inert liquid, for example an alcohol, water or the product of the particular reaction for which the catalyst is used. If appropriate, the catalyst must then be freed of the inert liquid before commencement of the actual reaction.

The storage of the catalyst under inert substances enables uncomplicated and safe handling and storage of the catalyst.

After the reduction, the catalyst can also be contacted with an oxygen-comprising gas stream such as air or a mixture of air with nitrogen.

This affords a passivated catalyst. The passivated catalyst generally has a protective oxide layer. This protective oxide layer simplifies the handling and storage of the catalyst, such that, for example, the installation of the passivated catalyst into the reactor is simplified. Before being contacted with the reactants, a passivated catalyst is preferably reduced as described above by treating the passivated catalyst with hydrogen or a hydrogen-comprising gas. The reduction conditions correspond generally to the reduction conditions which are employed in the reduction of the catalyst precursors. The activation generally eliminates the protective passivation layer.

3-Aminopropanol is obtained by reaction of ethylene cyanohydrin with hydrogen in the presence of ammonia, the reaction preferably being effected in the presence of one of the abovementioned catalysts.

The molar ratio of ammonia used to ethylene cyanohydrin used is typically within a range from 1:50 to 100:1, preferably 1:1 to 50:1, more preferably 1.1:1 to 25:1 and most preferably 2:1 to 10:1.

The reaction is generally performed at a pressure of 1 to 500 bar, preferably of 10 to 400 bar, particularly of 100 to 300 bar and most preferably of 120 to 250 bar. The pressure is maintained and controlled generally via the metered addition of hydrogen.

The hydrogenation of ethylene cyanohydrin to 3-aminopropanol is effected generally at temperatures of 20 to 400° C., preferably 20 to 250° C., more preferably 25 to 200° C. and most preferably 50 to 150° C.

The reaction of ethylene cyanohydrin with ammonia can be effected in substance or in the presence of a solvent, for example in ethers, such as methyl tert-butyl ether, ethyl tert-butyl ether or tetrahydrofuran (THF); alcohols such as methanol, ethanol or isopropanol; hydrocarbons such as hexane, heptane or raffinate cuts; aromatics such as toluene; amides such as dimethylformamide or dimethylacetamide, or lactams such as N-methylpyrrolidone, N-ethylpyrrolidone, N-methylcaprolactam or N-ethylcaprolactam. Useful solvents are also suitable mixtures of the solvents listed above. The solvent can be used in a proportion of 5 to 95% by weight, preferably 20 to 70%, more preferably 30 to 60%, based in each case on the total weight of the reaction mixture, the total weight of the reaction mixture being the sum of the masses of the starting materials and solvents used in the process.

Preference is given to using the 3-aminopropanol product of value as the solvent because this dispenses the removal of the solvent during the workup.

In a particularly preferred embodiment, the reaction of ethylene cyanohydrin with ammonia is performed in substance, i.e. without addition of solvent.

The process according to the invention can be performed continuously, batchwise or semibatchwise.

Preference is given to performing the process according to the invention in a high-pressure stirred tank reactor, fixed bed reactor or fluidized bed reactor.

In a particularly preferred embodiment, the process according to the invention is performed in one or more fixed bed reactors.

The fixed bed reactor can be operated either in liquid phase mode or in trickle mode. In the case of the preferred trickle mode, preference is given to using a liquid distributor for the reactor feed at the inlet of the reactor. When two reactors are used, both can be operated in liquid phase mode or trickle mode. Alternatively, the first reactor can be operated in liquid phase mode and the second reactor in trickle mode, or vice versa.

Ethylene cyanohydrin and ammonia can be introduced together into the reaction zone of the reactor, for example as a premixed reactant stream. The addition can also be effected separately, in which case the reactants are mixed at the inlet of the reactor in a continuous process, for example by means of liquid distributors or appropriate internals. In the case of batchwise performance, ethylene cyanohydrin and ammonia can be introduced into the reaction zone of the reactor simultaneously, at different times or successively.

The residence time in the batchwise hydrogenation of ethylene cyanohydrin is generally 15 minutes to 24 hours, preferably 30 minutes to 12 hours, more preferably 30 minutes to 6 hours.

In the case of performance in a preferably continuous process, the residence time is generally 0.1 second to 24 hours, preferably 1 minute to 10 hours, more preferably 15 minutes to 300 minutes and most preferably 15 minutes to 60 minutes.

For the preferred continuous processes, “residence time” in this context means the residence time over the catalyst, and thus the residence in the catalyst bed for a fixed bed catalyst; for fluidized bed reactors, the synthesis part of the reactor (part of the reactor where the catalyst is localized) is considered.

In the continuous reaction of ethylene cyanohydrin with ammonia, preference is given to establishing a catalyst hourly space velocity of 0.01 to 10 kg, preferably of 0.05 to 7 kg and more preferably of 0.1 to 5 kg of ethylene cyanohydrin per kg of catalyst and hour.

The reaction output which comprises 3-aminopropanol and is obtained in the reaction of ethylene cyanohydrin with hydrogen in the presence of ammonia comprises, as well as 3-aminopropanol, unconverted ethylene cyanohydrin, water, small amounts of by-products and unconverted ammonia.

The ammonia content of the reaction output from the hydrogenation reactor is, according to the amount of ammonia used, in the range from 1 to 90% by weight, preferably 5 to 80% by weight, more preferably 20 to 70% by weight and most preferably 40 to 70% by weight, based in each case on the mass of the reaction output.

The output from the hydrogenation reactor is worked up in accordance with the invention by distilling the reaction output in two or more stages.

In the context of the present invention, it has been found that a 3-aminopropanol which meets the strict quality demands of the cosmetic and pharmaceutical industry is obtainable only when the content of ammonia in the reaction output before introduction in the first distillation stage is 1% by weight or less, and the bottom temperature in the two distillation stages is not more than 135° C.

When the ammonia content of the reaction output from the hydrogenation reactor comprises more than 1% by weight of ammonia, based on the total mass of the reaction output, the ammonia content of the reaction output from the hydrogenation reactor has to be reduced to 1% by weight or less before introduction into the first distillation stage.

In a preferred embodiment, the ammonia content of the output from the hydrogenation reactor is reduced by introducing the reaction output from the hydrogenation reactor into a distillation column (ammonia removal).

The ammonia removal is effected preferably in a pressure column, the column pressure being selected such that the ammonia can be condensed with the cooling medium present at the given cooling medium temperature, for example cooling water.

The ammonia removal is effected preferably in a distillation column which has internals for increasing the separating performance.

The ammonia removal is more preferably performed in a tray column since such a column is very suitable for operation at high pressure.

In a tray column, intermediate trays are present in the interior of the column, on which the mass transfer takes place. Examples of different tray types are sieve trays, tunnel-cap trays, dual-flow trays, bubble-cap trays or valve trays.

The distillative internals may also be present as a structured packing, for example as a sheet metal packing, such as Mellapak 250 Y or Montz Pak, B1-250 type, or as a structured ceramic packing or as a random packing, for example of Pall rings, IMTP rings (from Koch-Glitsch), Raschig Superrings, etc. Structured or random packings may be arranged in one bed or preferably in a plurality of beds.

The exact operating conditions of the distillation column can be determined in a routine manner, according to the separating performance of the column used, by the person skilled in the art with reference to the known vapor pressures and evaporation equilibria of the components introduced into the distillation column by conventional calculation methods.

The reaction output from the hydrogenation reactor is preferably supplied in a spatial region between 30% and 90% of the theoretical plates of the distillation column (counted from the bottom), more preferably within a spatial region between 50% and 80% of the theoretical plates of the distillation column. For example, the feed may be somewhat above the middle of the theoretical plates. The optimal feed point can be determined by the person skilled in the art depending on the ammonia concentration with the customary calculation tools.

The number of theoretical plates is generally in the range from 5 to 30, preferably 10 to 20.

The top pressure is more preferably 1 to 30 bar, more preferably 10 to 25 bar and especially preferably 15 to 20 bar.

In column bottom, preference is given to establishing a temperature above the evaporation temperature of the ammonia, such that ammonia is converted completely or very substantially completely to the gas phase.

Particular preference is given to establishing a temperature which corresponds closely to the boiling temperature of the mixture to be removed via the bottom at column bottom pressure. The temperature depends on the type and composition of the substances present in the bottom product and can be determined by the person skilled in the art with the customary thermodynamic calculation tools.

Preference is given to establishing a temperature of 165 to 200° C., more preferably 175 to 195° C. and especially preferably 180 to 190° C. For example, it is possible with preference to establish a column bottom temperature of 185° C. at a column top pressure of 17 bar.

The condenser of the distillation column is generally operated at a temperature at which the predominant portion of the ammonia is condensed at the appropriate top pressure. In general, the operating temperature of the condenser is in the range from 25 to 70° C., preferably 25 to 45° C.

The return stream at the top of the column is generally established such that the predominant amount of 3-aminopropanol and water are retained within the column, such that they are obtained virtually completely as the bottom product. The condensate obtained in the condenser is preferably recycled to an extent of less then 50%, preferably to an extent of less than 25%, into the top of the distillation column.

The energy required for the evaporation is typically introduced by an evaporator in the column bottom.

In the condenser, the condensate obtained is predominantly ammonia.

The ammonia obtained as the condensate can, after a purification or preferably directly, be used as a starting material for further chemical syntheses. For example, the ammonia obtained as the condensate can be reused for preparation of 3-aminopropanol, by recycling the ammonia to the 3-aminopropanol preparation process.

The bottom output obtained from the ammonia removal is generally a mixture which comprises 3-aminopropanol, water and generally relatively high-boiling amines, and also organic by-products.

In addition, the bottom output from the ammonia removal generally comprises less than 10% by weight and preferably less than 5% by weight of residual ammonia.

When the output from the ammonia removal has an ammonia content of 1% by weight or less, preferably 0.5% by weight or less, more preferably 0.25% by weight or less and especially preferably 0.1% by weight or less, the output from the ammonia removal can be introduced directly as feed into the first distillation stage.

In a very particularly preferred embodiment, the ammonia content of the output from the ammonia removal is, however, reduced further by degassing (ammonia degassing).

For the degassing, the reaction output comprising 3-aminopropanol can optionally be decompressed, heated and/or treated with a stripping gas.

The degassing of ammonia is preferably effected in a degassing column.

The degassing can be effected, for example, in an apparatus customary for that purpose, as described, for example, in: Kirk-Othmer, Encyclopedia of Chemical Technology, 3rd ed., Vol. 7, John Wiley & Sons, New York, 1979, pages 870-881, such as evaporation still or rectification column, for example sieve tray column, bubble-cap tray column, column with structured packing or column with random packing.

The reaction output from the ammonia removal which comprises 3-aminopropanol preferably degassed in a distillation column with stripping and rectifying sections, in which case the reaction output comprising the 3-aminopropanol is preferably fed in in the upper region of the column, and the ammonia-depleted reaction output is drawn off at the bottom of the column and can then be fed in accordance with the invention to a two-stage or multistage distillation.

At the top of the column, a gaseous stream is generally drawn off, which comprises essentially ammonia.

The exact operating conditions of the degassing column can be determined in a routine manner according to the separating performance of the column used by the person skilled in the art with reference to the known vapor pressure and evaporation equilibria of the components present in the reaction output comprising 3-aminopropanol, by conventional calculation methods.

The ammonia degassing is effected preferably in a distillation column which has internals for increasing the separating performance.

The ammonia degassing is more preferably performed in a tray column. In a tray column, intermediate trays are present in the interior of the column, on which the mass transfer takes place. Examples of different tray types are sieve trays, tunnel-cap trays, dual-flow trays, bubble-cap trays or valve trays.

In a further preferred embodiment, the distillative internals may also be present as a structured packing, for example as a sheet metal packing, such as Mellapak 250 Y or Montz Pak, B1-250 type, or as a structured ceramic packing or as a random packing, for example of Pall rings, IMTP rings (from Koch-Glitsch), Raschig Superrings, etc. Structured or random packings may be arranged in one bed or preferably in a plurality of beds.

The crude product from the ammonia removal is preferably supplied in a spatial region between 50% and 90% of the theoretical plates of the distillation column (counted from the bottom), more preferably in a spatial region between 60% and 85% of the theoretical plates of the distillation column. For example, the supply may be above the middle of the theoretical plates. The optimal feed point can be determined by the person skilled in the art as a function of the ammonia concentration with the customary calculation tools.

The number of theoretical plates is generally in the range from 10 to 100, preferably 15 to 80, more preferably 20 to 70 and most preferably 25 to 60.

The top pressure is more preferably 500 to 3000 mbar, more preferably 800 to 2000 mbar and most preferably 1000 to 1500 mbar.

In the bottom of the column, preference is given to establishing a temperature above the evaporation temperature of the ammonia, such that ammonia is converted completely or very substantially to the gas phase.

Particular preference is given to establishing a temperature which does not exceed 135° C., preferably 130° C. and more preferably 125° C.

For example, it is possible with preference to establish a column bottom temperature of 135° C. at a column top pressure of 1013 mbar.

The energy required for the evaporation is typically introduced by an evaporator in the column bottom.

In a preferred embodiment, a stripping gas is introduced into the column. Stripping gases are gases which behave inertly under the present reaction conditions and do not react with the substances present in the reaction mixture. The stripping gases used may be inert gases, such as nitrogen or noble gases (helium, neon, argon, xenon). Preference is given to using nitrogen as an inert gas.

Stripping gas is preferably introduced into the lower region of the distillation column and thus conducted in countercurrent to the liquid stream.

The introduction can be effected into the bottom of the column, for example by means of a distributor ring or of a nozzle, but it can also be effected into the lower region of the distillation column, preferably into a spatial region up to 30%, preferably up to 20% and more preferably up to 10% of the theoretical plates of the distillation column (counted from the bottom). The stripping gas introduced is generally mixed thoroughly with the liquid flowing in the opposite direction by the internals present in the column.

The flow of inert gas supplied is preferably 0.001 to 1 m³/h, more preferably 0.005 to 0.1 m³/h and most preferably 0.01 to 0.05 m³/h of inert gas per kg/h of feed.

In the upper region of the column, ammonia is generally drawn off as a gaseous stream.

The ammonia obtained can be used as a starting material for further chemical syntheses after a purification or preferably directly; for example, the ammonia obtained can be recycled into the preparation process.

As the bottom output from the ammonia degassing, a mixture comprising 3-aminopropanol, residual ammonia, water and possibly relatively high-boiling secondary components is generally obtained.

In the lower region of the degassing column, an output is generally obtained which has an ammonia content in the range from 0.001 to 1% by weight, preferably 0.005 to 0.5% by weight, more preferably in the range from 0.01 to 0.25% by weight and especially preferably in the range from 0.015 to 0.1% by weight, based on the total mass of the stream comprising 3-aminopropanol.

The ammonia degassing, i.e. the degassing of the output comprising 3-aminopropanol from the ammonia removal, can, however, also be effected by introducing a stripping gas, preferably nitrogen, into a storage vessel or a tank reactor. The stripping gas can be introduced by means of a distributor nozzle or of a distributor ring. Since the vapor pressure of ammonia in the liquid phase is higher than in the gas, the ammonia is transferred to the gas phase.

The ammonia-containing offgas from the stripping operation is generally worked up or sent to disposal.

The output from the ammonia degassing, for example the output from the degassing column or the contents of the stripped storage tank or reactor, can be introduced as feed into the two-stage or multistage distillation as the feed stream.

When the ammonia content of the output comprising 3-aminopropanol from the ammonia degassing is more than 1% by weight, preferably more than 0.5% by weight, more preferably more than 0.25% by weight and especially preferably more than 0.1% by weight, the output from the degassing columns should, however, be subjected to a further degassing step in order to further reduce the ammonia content before it is introduced into the inventive workup.

The feed stream which is introduced into the two-stage or multistage distillation generally comprises 3-aminopropanol, residual ammonia, water and optionally relatively high-boiling secondary components.

According to the invention, the feed stream which is introduced into the inventive two-stage or multistage distillation has an ammonia content of 1% by weight or less, based on the total mass of the feed stream. The ammonia content should preferably be less than 0.5% by weight, more preferably less than 0.25% by weight and most preferably less than 0.1% by weight.

In general, the ammonia content of the feed stream should be in the range from 0.001 to 1% by weight, preferably 0.005 to 0.5% by weight, more preferably in the range of 0.01 to 0.25% by weight and especially preferably in the range from 0.015 to 0.1% by weight.

As described above, the ammonia content in a feed which has an ammonia content of more than 1% by weight, preferably more than 0.5% by weight, more preferably more than 0.25% by weight and especially preferably more than 0.1% by weight should be reduced, for example by the above-described ammonia degassing and/or the ammonia removal.

The first stage of the distillation (water removal) is preferably effected in a distillation column, for example a sieve tray column, bubble-cap tray column, column with structured packing or column with random packing.

The crude aminopropanol is more preferably distilled in a rectification column with stripping and rectifying sections, in which case the crude aminopropanol is preferably fed in in the region of the middle of the column and a high boiler mixture is drawn off at the bottom of the column, which comprises predominantly aminopropanol and optionally relatively high-boiling by-products. At the top of the column, a liquid or gaseous stream is drawn off, which comprises essentially water and residues of ammonia.

The exact operating conditions can be determined in a routine manner, according to the separating performance of the column used, by the person skilled in the art with reference to the known vapor pressures and evaporation equilibria of the components present in the crude aminopropanol, by conventional calculation methods.

The distillation column preferably has internals for increasing the separating performance. The distillative internals may preferably be present as a structured packing, for example as a sheet metal packing such as Mellapak 250 Y or Montz Pak, B1-250 type. It is also possible for a packing with relatively low or increased specific surface area to be present, or it is possible to use a fabric packing or a packing with different geometry, such as Mellapak 252.Y. The advantages of the use of these distillative internals are the low pressure drop and the low specific liquid holdup compared to valve trays, for example. The internals may be present in one or more beds.

The feed stream which comprises 3-aminopropanol is preferably supplied in a spatial region between 25% and 75% of the theoretical plates of the distillation column (counted from the bottom), more preferably in a spatial region between 30% and 65% of the theoretical plates of the distillation column. For example, the feed may be somewhat below the middle of the theoretical plates. The optimal feed point can be determined by the person skilled in the art with the customary calculation tools.

The number of the theoretical plates is generally in the range from 5 to 50, preferably 20 to 40.

The top pressure is preferably 5 to 1000 mbar, more preferably 10 to 500 mbar, especially preferably 15 to 100 mbar.

In the column bottom, preference is given to establishing a temperature which is above the evaporation temperature of water but below the evaporation temperature of 3-aminopropanol.

According to the invention, the temperature in the bottom of the column is, however, not more than 135° C. The temperature in the bottom of the column is preferably 50 to 130° C., more preferably from 80 to 125° C. and especially preferably 100 to 125° C.

For example, it is possible with preference to establish a column bottom temperature of 130° C. at a column top pressure of 0.1 bar.

The condenser of the distillation column is generally operated at a temperature at which the predominant portion of the water is condensed at the corresponding top pressure. In general, the operating temperature of the condenser is in the range from 25 to 70° C., preferably 30 to 50° C.

The condensate obtained in the condenser is preferably recycled into the top of the distillation column to an extent of more than 30%, preferably to an extent of more than 40%.

The energy required for the evaporation is typically introduced by an evaporator in the column bottom.

In the condenser, a condensate which comprises predominantly water and residual ammonia is obtained.

In the bottom output, a mixture is generally obtained which comprises 3-aminopropanol and possibly higher by-products.

In the context of the present invention, the bottom output from the first distillation stage is referred to as “output of the first distillation stage”.

The output of the first distillation stage (water removal) is, in accordance with the invention, supplied to at least one further distillation stage (purifying distillation).

The second stage of the distillation is preferably likewise effected in a distillation column, for example sieve tray column, bubble-cap tray column, column with structured packing or column with random packing.

More preferably, the output of the first distillation stage is distilled in a rectification column with stripping and rectifying sections, in which case the output of the first distillation stage is preferably fed in in the region of the middle of the column, and a high boiler mixture which comprises predominantly unconverted ethylene cyanohydrin is drawn off at the bottom of the column. At the top of the column, a liquid or gaseous stream is drawn off, which comprises essentially pure aminopropanol.

The exact operating conditions can be determined in a routine manner, according to the separating performance of the column used, by the person skilled in the art with reference to the known vapor pressures and evaporation equilibria of the components present in the output of the first distillation stage, by conventional calculation methods.

The distillation column preferably has internals for increasing the separating performance. The distillative internals may preferably be present as a structured packing, for example as a sheet metal packing such as Mellapak 250 Y or Montz Pak, B1-250 type. It is also possible for a structured packing with relatively low or increased specific surface area to be present, or it is possible to use a fabric packing or a packing with different geometry such as Mellapak 252.Y. Advantages in the case of use of these distillative internals are the low pressure drop and the low specific liquid holdup compared to valve trays, for example. The internals may be present in one or more beds.

The output of the first distillation stage, which comprises 3-aminopropanol and possibly higher-boiling secondary components, is preferably supplied in a spatial region between 25% and 75% of the theoretical plates of the distillation column (counted from the bottom), more preferably in a spatial region between 30% and 65% of the theoretical plates of the distillation column. For example, the feed may be somewhat below the middle of the theoretical plates. The optimal feed point can be determined by the person skilled in the art with the customary calculation tools.

The number of theoretical plates is generally in the range from 5 to 100, preferably 30 to 80.

The top pressure is preferably 5 to 1000 mbar, more preferably 10 to 500 mbar, especially preferably 15 to 100 mbar.

In the bottom of the column, preference is given to establishing a temperature above the evaporation temperature of 3-aminopropanol.

According to the invention, the temperature in the bottom of the column is, however, not more than 135° C. The temperature in the bottom of the column is preferably 50 to 130° C., more preferably from 80 to 125° C. and especially preferably 100 to 125° C.

For example, a column bottom temperature of 120° C. can be established with preference at a column top pressure of 40 mbar.

For example, a column bottom temperature of 125° C. can be established with preference at a column top pressure of 70 mbar.

The condenser of the distillation column is generally operated at a temperature at which the predominant portion of the 3-aminopropanol is condensed at the corresponding top pressure. In general, the operating temperature of the condenser is in the range from 25 to 70° C., preferably 30 to 50° C.

Preferably, the condensate obtained in the condenser is recycled into the top of the distillation column to an extent of more than 80%, preferably to an extent of more than 90%.

The energy required for the evaporation is typically introduced by an evaporator in the column bottom.

In the bottom output, a mixture is generally obtained which comprises the relatively high-boiling secondary components.

The 3-aminopropanol obtained as the condensate of the second distillation stage generally need not be subjected to any further distillation stage, but can if required be worked up by distillation in one or more further stages.

The 3-aminopropanol obtained as the top output of the second distillation stage is, however, preferably not worked up any further.

The 3-aminopropanol obtained as the top output of the second distillation stage preferably has a purity of more than 99% by weight, more preferably more than 99.5% by weight, more preferably more than 99.7% by weight and especially preferably more than 99.9% by weight.

The 3-aminopropanol obtainable in accordance with the invention can be used for the preparation of 3-aminopropanol derivatives. More particularly, the 3-aminopropanol obtainable in accordance with the invention is suitable for preparation of products for cosmetic and/or therapeutic uses, especially panthenol, acambrosate, mefenorex, domperidon, ifosamid or urapidil.

The active ingredient panthenol is used by many manufacturers as an ingredient for skin creams and ointments, or else for lozenges, nasal sprays, eye drops and contact lens cleaning products.

Accordingly, the present invention also provides a process for preparing 3-aminopropanol derivatives, especially panthenol, acambrosate, mefenorex, domperidon, ifosamid and urapidil, wherein a 3-aminopropanol which is prepared in a process according to the invention is used in the preparation.

The 3-aminopropanol obtained by the process according to the invention has a higher purity than a 3-aminopropanol obtained by known distillation processes.

More particularly, the inventive 3-aminopropanol has only a low intrinsic odor, and so it is suitable as a starting material for the production of ointments, which are generally applied directly to the human skin.

The 3-aminopropanol obtainable in accordance with the invention meets the strict and high quality standards of the cosmetic and pharmaceutical industry.

The invention is illustrated in detail by the examples which follow.

### EXAMPLES

### General Methods:

### Example 1

### Preparation of 3-aminopropanol

Ethylene cyanohydrin (450 kg/h) was converted together with ammonia (850 kg/h) in the presence of hydrogen at a pressure of 180 bar and a temperature of 100° C. in a tubular reactor. The catalyst used was a catalyst according to Example A of EP-A-0742045. The catalyst hourly space velocity was 0.3 kg of ECHD/kg of catalyst/hour.

The reaction output was introduced into a distillation column which was operated at column top pressure of 17 bar (ammonia removal). The distillation column had 12 theoretical plates. The feed point was in the region of the 10th plate. The bottom temperature was 185° C.

The output from the ammonia removal was analyzed by gas chromatography and comprised: 93 area % of 3-aminopropanol;

2.5 area % of ammonia;

3.0 area % of dihydroxypropylamine

0.3 area % of diaminopropyl ether

0.2 area % of ethanediol

### Example 2

### Ammonia Degassing

The reaction output from Example 1 was introduced into a degassing column. The degassing column had 50 theoretical plates. The feed point was in the region of the 25th plate. The feed was 1600 kg/h. The distillation was operated at a pressure of 1 bar abs. and a bottom temperature of 130° C. The stripping gas used was nitrogen, which was fed in via the lower region of the columns. The flow rate of nitrogen supplied was 30 m³/h.

The output from the degassing column was analyzed by gas chromatography and comprised:

94.08 area % of 3-aminopropanol

0.07 area % of ammonia

0.83% water

### Example 3

### Two-Stage Distillation

The output from the ammonia degassing (Example 2) was introduced into a two-stage distillation. The first distillation column (water removal) had 33 theoretical plates. The feed point was in the region of the 20th plate. The column top pressure was 70 mbar abs. The column bottom temperature was 120° C. At the top of the column, water and residues of ammonia were condensed. The output at the bottom of the column comprised 3-aminopropanol and higher-boiling by-products (crude aminopropanol).

The composition of the bottom output was analyzed by gas chromatography and was:

96.0 area % of 3-aminopropanol

0.8 area % of ethanediol

The bottom output from the water removal was passed into a further distillation column (purifying distillation). This second distillation column (purifying distillation) had 62 theoretical plates. The feed point was in the region of the 40th plate. The column top pressure was 40 mbar. The column bottom temperature was 122° C. At the top of the column, pure 3-aminopropanol was distilled. The output at the bottom of the column comprised higher-boiling by-products.

The composition of the top output was analyzed by gas chromatography and was:

99.95 area % of 3-aminopropanol.

### Example 4

### Two-Stage Distillation

The output from the 3-aminopropanol preparation (Example 1) was introduced directly into a two-stage distillation. The first distillation column (water removal) had 33 theoretical plates. The feed point was in the region of the 20th plate. The column top pressure was 350 mbar abs. The column bottom temperature was 158° C. At the top of the column, water and residues of ammonia were condensed. The output at the bottom of the column comprised 3-aminopropanol and higher-boiling by-products (crude aminopropanol).

The composition of the bottom output was analyzed by gas chromatography and was: 98.2 area % of 3-aminopropanol.

The bottom output from the water removal was passed into a further distillation column (purifying distillation). This second distillation column (purifying distillation) had 62 theoretical plates. The feed point was in the region of the 40th plate. The column top pressure was 180 mbar. The column bottom temperature was 149° C. At the top of the column, pure 3-aminopropanol was distilled. The output at the bottom of the column comprised higher-boiling by-products.

The composition of the top output was analyzed by gas chromatography and was: 99.8 area % of 3-aminopropanol.

### Example 5

### Ammonia Degassing

The reaction output from Example 1 was introduced into a degassing column. The degassing column had 33 theoretical plates. The feed point was in the region of the 20th plate. The feed was 3000 kg/h. The distillation was operated at a pressure of 85 mbar abs. and a bottom temperature of 127° C. The stripping gas used was nitrogen, which was fed in via the lower region of the columns. The flow rate of nitrogen supplied was 30 m³/h.

The output from the degassing column was analyzed by gas chromatography and comprised:

95.7 area % of 3-aminopropanol

0.02 area % of ammonia

### Example 6

### Two-Stage Distillation

The output from the degassing column (Example 2) was introduced into a two-stage distillation. The first distillation column (water removal) had 33 theoretical plates. The feed point was in the region of the 20th plate. The column top pressure was 70 mbar abs. The column bottom temperature was 118° C. At the top of the column, water and residues of ammonia were condensed. The output at the top of the column comprised 3-aminopropanol and higher-boiling by-products (crude aminopropanol).

The composition of the bottom output was analyzed by gas chromatography and was: 95.5 area % of 3-aminopropanol

0.8 area % of ethanediol

The bottom output from the water removal was passed into a further distillation column (purifying distillation). This second distillation column (purifying distillation) had 62 theoretical plates. The feed point was in the region of the 40th plate. The column top pressure was 40 mbar. The column bottom temperature was 120° C. At the top of the column, pure 3-aminopropanol was distilled. The output at the bottom of the column comprised higher-boiling by-products.

The composition of the top output was analyzed by gas chromatography and was: 99.95 area % of 3-aminopropanol;

The outputs from the examples were subjected to an olfactory assessment.

For this purpose, the 3-aminopropanol obtained in Examples 3, 4 and 6 was converted to panthenol, the odor of which was then assessed. In the odor assessment, the following protocol was employed:

Preparation of Panthenol:

A 1 l four-neck flask was initially charged with 150 g of 3-aminopropanol. While stirring, 260 g of D-pantolactone were added slowly at room temperature. After the addition had ended, the reaction mixture was heated to 60° C. and stirred for a further 5 hours. The D-pantolactone used was washed twice beforehand with methyl tert-butyl ether (MTBE) and then dried.

The crude panthenol obtained by the reaction of 3-aminopropanol and D-pantolactone was subsequently degassed and distilled.

The degassing was performed in a thin-film evaporator at a pressure of 0.027 mbar, a bottom temperature of 80° C. and a lamellar speed of 280 rpm. After the degassing, the apparatus was cleaned by repeatedly purging with demineralized water and 2-propanol with subsequent drying under reduced pressure.

Subsequently, the degassed panthenol was distilled. The distillation was performed in the same apparatus in which the degassing had already been undertaken. The bottom temperature was 120° C., the pressure 0.027 mbar and the lamellar speed 800 rpm.

In the internal condenser, at a cooling coil temperature of 60° C., panthenol was obtained.

75 g of the distilled panthenol were homogenized for 24.1 g of distilled water at 40° C. The aqueous panthenol solution thus prepared is referred to hereinafter as test sample.

The panthenol thus obtained (test sample) was assessed olfactorily.

Olfactory Assessment:

The sensory test was carried out in a single determination or, in the case of doubt, in a repeat determination by trained personnel using a validated method. In the course of the validation, in a practical model case, a significance level=0.05 (statistical evaluation process by the binomial theorem) was preset and confirmed in 9-fold repetition.

3 ml of the test sample were pipetted into a sample bottle (diameter 70 mm, height 120 mm, capacity 370 ml) with a disposable polyethylene pipette (Makro 155, graduated up to 3.0 ml).

In a further sample bottle, a reference sample of acceptable odor was prepared in the same way as a standard.

Sample bottles were closed and conditioned (10 minutes at room temperature). This ensures that an equilibrium between liquid phase and the gas phase enriched with volatile constituents can be established. The conditioning times should be maintained with a tolerance of +/−1 minute.

After the conditioning had been completed, the tester opened the sample bottle of the standard, took in the odor of the headspace and closed the bottle again. Without delay, the bottle of the sample specimen was then opened and smelled in the same way and closed again thereafter.

Before a repeat measurement, the samples were conditioned again.

If the tester detected no odor difference from the standard, the sample was assessed with the rating “ok” or “yes”.

If product-untypical deviations from the standard were found, which put the intended end use into question, the sample was assessed with the rating “oos” or “no”.

TABLE 1
Result of the olfactory assessment:

| | | | | Bottom temp. | |
|---|---|---|---|---|---|
| | NH₃ content | Bottom | Bottom | of the second | |
| | of the feed | temp. in | temp. of the | dist. stage | Olfactory |
| Ex- | which is supplied | NH₃ | first dist. stage | (purifying | assess- |
| ample | to the workup | degassing | (water removal) | distillation) | ment |
| 3 | 0.02 | 130 | 120 | 122 | ok |
| 4 | 2.5 | — | 158 | 149 | oos |
| 6 | 0.02 | 127 | 118 | 120 | ok |

1-14. (canceled)

15. A process for purifying a reaction output which comprises 3-aminopropanol and is obtained by reacting ethylene cyanohydrin with hydrogen in the presence of ammonia, which comprises distilling the reaction output comprising 3-aminopropanol in two or more stages, the ammonia content of the reaction output comprising 3-aminopropanol before introduction into the first distillation stage being 1% by weight or less and the temperature in the distillation stages being not more than 135° C.

16. The process according to claim 15, wherein the feedstream which comprises 3-aminopropanol and is introduced into the first distillation stage has an ammonia content of 0.1% by weight or less.

17. The process according to claim 15, wherein ethylene cyanohydrin is prepared by reaction of ethylene oxide and hydrogen cyanide.

18. The process according to claim 15, wherein ethylene cyanohydrin is reacted with hydrogen in the presence of ammonia in the presence of a catalyst which is obtained by reduction of a catalyst precursor.

19. The process according to claim 18, wherein the catalyst precursor comprises CoO, NiO, CuO, RuO(OH)ₓ or LiCoO₂ as catalytically active components.

20. The process according to claim 19, wherein the catalytically active mass of the catalyst precursor, before it is reduced with hydrogen, comprises 55 to 98% by weight of Co, calculated as CoO, 0.2 to 15% by weight of phosphorus, calculated as H₃PO₄, 0.2 to 15% by weight of manganese, calculated as MnO₂, and 0.2 to 15% by weight of alkali metal, calculated as M₂O (M=alkali metal).

21. The process according to claim 15, wherein ethylene cyanohydrin is reacted with hydrogen in the presence of ammonia in a fixed bed reactor.

22. The process according to claim 15, wherein the molar ratio of ammonia used to ethylene cyanohydrin used is within a range from 1:1 to 50:1.

23. The process according to claim 15, wherein the ammonia content of the reaction output comprising 3-aminopropanol before it is fed into the first distillation stage is reduced by degassing.

24. The process according to claim 23, wherein the degassing is effected in a rectification column with introduction of stripping gas.

25. The process according to claim 15, wherein the bottom temperature in the first and/or second distillation stage is 100 to 125° C.

26. A process for preparing 3-aminopropanol by reacting ethylene cyanohydrin with hydrogen in the presence of ammonia, which comprises performing the purification of the reaction output comprising 3-aminopropanol according to claim 15.

27. A process for preparing 3-aminopropanol derivatives, which comprises preparing the 3-aminopropanol used according to claim 15.

28. A process for preparing panthenol, acambrosate, mefenorex, domperidon, ifosamid or urapidil, which comprises preparing the 3-aminopropanol used according to claim 15.
